(19) United States
(12) Patent Application Publication
(10) Pub. No.: US 2024/0164834 A1
(43) Pub. Date: May 23, 2024

(54) SYSTEMS AND METHODS FOR CATHETERS HAVING COMBINED MAPPING AND ABLATION CAPABILITIES

(71) Applicant: St. Jude Medical, Cardiology Division, Inc., St. Paul, MN (US)

(72) Inventors: Greg Olson, Elk River, MN (US); Isaac Remer, Richfield, MN (US); Jodee Wakefield, Golden Valley, MN (US); Derek Sutermeister, Ham Lake, MN (US); Troy T. Tegg, Elk River, MN (US); Patrick K. Kasi, Woodbury, MN (US); D. Curtis Deno, Andover, MN (US)

(21) Appl. No.: 18/506,524

(22) Filed: Nov. 10, 2023

Related U.S. Application Data
(60) Provisional application No. 63/426,533, filed on Nov. 18, 2022.
(60) Provisional application No. 63/533,003, filed on Aug. 16, 2023.

(51) Int. Cl. A61B 18/14; A61B 5/00; A61B 5/287; A61B 5/367; A61B 18/12

(52) U.S. Cl. CPC A61B 18/1492; A61B 5/287; A61B 5/367; A61B 5/6858; A61B 18/1206; A61B 2018/00839

(57) ABSTRACT

A catheter assembly is provided. The catheter assembly includes a tip electrode array including at least one tip electrode, the tip electrode array located at a distal end of the catheter assembly, and a mini electrode array including a plurality of mini electrodes, the mini electrode array positioned proximal of the tip electrode array, wherein each of the at least one tip electrode and each of the plurality of mini electrodes are configured to be activated independent of one another for mapping applications, and wherein at least some of the at least one tip electrode and the plurality of mini electrodes are configured to be activated in unison for ablation applications.

### CROSS-REFERENCE TO RELATED APPLICATIONS

This application claims priority to U.S. Provisional Patent Application No. 63/426,533, filed Nov. 18, 2022, and U.S. Provisional Patent Application No. 63/533,003, filed Aug. 16, 2023, both of which are incorporated by reference herein in their entirety.

### FIELD OF THE DISCLOSURE

The present disclosure relates generally to tissue ablation systems. In particular, the present disclosure relates to catheters that are capable of use for both mapping and ablation applications.

### BACKGROUND

It is generally known that ablation therapy may be used to treat various conditions afflicting the human anatomy. For example, ablation therapy may be used in the treatment of atrial arrhythmias. When tissue is ablated, or at least subjected to ablative energy generated by an ablation generator and delivered by an ablation catheter, lesions form in the tissue. Electrodes mounted on or in ablation catheters are used to cause cell death (e.g., via tissue apoptosis or necrosis) in cardiac tissue to correct conditions such as atrial arrhythmia (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a non-thermal ablation technique that involves applying strong electric-fields that induce pore formation in the cellular membrane. The electric field may be induced by applying a relatively short duration pulse which may last, for instance, from a nanosecond to several milliseconds. Such a pulse may be repeated to form a pulse train. When such an electric field is applied to tissue in an in vivo setting, the cells in the tissue are subjected to trans-membrane potential, which opens the pores on the cell wall. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

Catheter-based cardiac mapping and catheter-based ablation are established diagnostic and therapeutic strategies for a range of cardiac arrhythmias. Many electrophysiology procedures are carried out using minimally invasive surgical methods, wherein one or more instruments are inserted through one or more small incisions into a patient's body. An instrument can have a fixed profile or variable profile (e.g., a variable diameter loop). Mapping or imaging systems typically compute metrics from EGM signals and provide a visual display of EGM metrics (color gradient) of the endocardial surface.

With respect to ablation, an instrument may also include a rigid or flexible component having an ablation device at or near its distal end that is generally positioned adjacent to the tissue to be ablated. Pulse field, radio frequency, microwave energy, laser energy, extreme heat, and extreme cold can be delivered by the ablation device to necrotize a tissue. Catheter-based devices have been invaluable for various medical and surgical applications because they are minimally invasive and allow for precise treatment of localized discrete tissues that are otherwise inaccessible.

Catheter-based imaging (i.e., mapping) systems are well known in the field of cardiac electrophysiology. Omnipolar mapping technology (OT) has made it possible to reliably process information that underlies cardiac rhythms. Specifically, OT is a signal processing approach that utilizes electrical signals from cardiac electrophysiology (EP) catheters more completely. OT acknowledges directional properties of intracardiac EGMs resolved by multielectrode catheters and software algorithms to characterize cardiac electrical activity in a manner that is insensitive to catheter-wavefront orientation. The resulting information is presented in a manner that conforms to anatomic and physiologic directions instead of strictly from a catheter's perspective.

Radio frequency (RF)-based ablation systems are known in the field of cardiac electrophysiology. During RF ablation procedures, a specially designed probe is typically positioned directly into a patient's target region. Once a physician has performed a diagnostic EP study, they would then insert an ablation catheter that is especially designed to administer radio frequency energy to a specific region of interest within a patient's heart. Most ablation catheters are quadripolar with a larger distal tip that contains a mechanism that delivers the RF energy to the heart. RF energy may be delivered as alternating electrical current typically in the frequency range of 350-750 kilohertz (kHz) in order to accelerate the electrons in the cardiac cells-generating heat which destroys the cells within a certain range of the catheter tip.

Like diagnostic catheters, there are many different types of ablation catheters. These various types of catheters are designed to aid the physician in ablating different locations in patients of varying sizes. It is common for physicians to choose an ablation catheter of their preference. However, a challenging case may prompt the doctor to switch to other types of catheters. The ablation catheter delivers energy to the heart to destroy cells that may be causing the patient's arrhythmia. This is commonly achieved using an RF generator. Energy from the generator is sent through a connecting cable to the ablation catheter where it is focused on a specific site within the patient's heart. The goal is to form a small, discreet scar at the selected site. Once formed, the scar prevents the transmission of electrical signals through that region and hopefully the arrhythmia terminates.

When the ablation system is set up, either the temperature or the power control must be selected. Generally, physicians use temperature control in which a desired temperature is selected and programmed into the generator. The maximum power and duration of individual ablation trials, also known as “burns” are selected. When these parameters are all entered, the ablation process is ready to begin.

Upon activation of the ablation catheter, continuous readings of power display, temperature, impedance, and time are displayed. The physician must be continuously informed of the above-mentioned value metrics. Upon any significant change in one of the parameters, a technician must let the physician know immediately. In this way, any adverse effects during the ablation procedure can be avoided. Ablation procedures offer many advantages over open surgical procedures. Patients are often unable to be treated with conventional surgical techniques. Moreover, it can be debilitating when a patient must undergo a second or larger surgical procedure. Ablation can be performed multiple times on different occasions without risks associated with a surgical procedure.

One type of ablation procedure, an emerging technology with potential advantages, is known pulsed field ablation PFA. While in the early years direct current (DC) shock therapy was abandoned due to safety concerns, it became known in the art that DC shocks provided irreversible electroporation (IRE). The mechanism of lesion formation in IRE is a function of electric field exposure that breaks down cell membrane permeability, leading to cell death. Pulsed field ablation is a form of IRE that employs (most commonly) trains of bipolar and biphasic high voltage and very-short duration pulses that result in destabilization of the cellular membranes (formation of pores in the cytoplasmic membrane) and death by a mechanism of irreversible electroporation. This method has several potential advantages for ablation of cardiac arrhythmias, including higher selectivity to myocardial tissue and smaller thermal effect, reducing the risk for inadvertent injury of blood vessels, nerves, and the esophagus.

It is known in the cardiac electrophysiology art that PFA can produce transmural and durable atrial lesions with minimal effect to the esophagus, phrenic nerve, and coronary arteries. As a consequence, there is heightened interest in PFA as an alternative to RF ablation, especially for atrial fibrillation treatment. Among known PFA ablation catheter designs, the majority are stand-alone ablation catheters without mapping capabilities or integration with electroanatomic mapping systems.

While different methods of ablation have their respective advantages, there may be certain circumstances where one method is desired over the other. Furthermore, circumstances may change rapidly during an ablation procedure, requiring that an ablation catheter be quickly replaced by a mapping catheter, another type of ablation catheter, and vice-versa. There is presently no system available that can provide a medical technician with the freedom to assess electrogram signals independent of catheter orientation and select an ablation technique based on the patient's current medical condition and perform sequential or simultaneous ablation procedures without the difficulty and time-consuming effort of constantly replacing one type of catheter with another.

Accordingly, given the need to shorten procedure times and the need for precision during ablation procedures, it would be desirable to provide an ablation system that allows for catheter-orientation independence, wave-speed measurement, and direction of propagation, RF energy, cryoablation, and PFA. For example, a real-time assessment of a specific target tissue area may be followed by a cryoablation and RF ablation; or cryoablation and then PFA. It is also desirable to provide an integrated ablation system that provides an accurate view of the underlying mechanisms especially when operating on regions that are difficult to access area such as the Atrioventricular (AN) node.

Some practitioners of electrophysiology have advocated for use of technologies that combine various modalities as a way to overcome certain shortcomings of individual treatment approaches. The ability to apply therapy without the need to remove one diagnostic catheter and then guess where to position an ablation catheter could potentially create a very flexible, safe, and efficient instrument that will greatly improve a physician workflow, as well as reduce time needed to administer therapy to patients.

### BRIEF SUMMARY OF THE DISCLOSURE

In one aspect, a catheter assembly is provided. The catheter assembly includes a tip electrode array including at least one tip electrode, the tip electrode array located at a distal end of the catheter assembly, and a mini electrode array including a plurality of mini electrodes, the mini electrode array positioned proximal of the tip electrode array, wherein each of the at least one tip electrode and each of the plurality of mini electrodes are configured to be activated independent of one another for mapping applications, and wherein at least some of the at least one tip electrode and the plurality of mini electrodes are configured to be activated in unison for ablation applications.

In another aspect, an electroporation system is provided. The electroporation system includes a generator, and a catheter coupled to the generator, the catheter including a handle, a shaft extending distally from the handle, and a catheter assembly coupled to a distal end of the shaft. The catheter assembly includes a tip electrode array including at least one tip electrode, the tip electrode array located at a distal end of the catheter assembly, and a mini electrode array including a plurality of mini electrodes, the mini electrode array positioned proximal of the tip electrode array, wherein each of the at least one tip electrode and each of the plurality of mini electrodes are configured to be activated independent of one another for mapping applications, and wherein at least some of the at least one tip electrode and the plurality of mini electrodes are configured to be activated in unison for ablation applications.

In yet another aspect, an ablation system is provided. The ablation system includes a console, at least one catheter, a cable system coupling the console to the at least one catheter, a hub coupled between the console and the at least one catheter, a first generator coupled to the hub, the first generator configured to deliver a first type of ablation energy to the at least one catheter via the hub, and a second generator coupled to the hub, the second generator configured to deliver a second type of ablation energy to the at least one catheter via the hub, wherein the console is configured to selectively control the delivery of the first and second types of ablation energy to the at least one catheter via the hub.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIG. 2 is a perspective view of one embodiment of a catheter assembly that may be used with the system shown in FIG. 1.

FIG. 3A is a perspective view of an alternative embodiment of a catheter assembly that may be used with the system shown in FIG. 1.

FIG. 3B is a perspective view of an alternative embodiment of a catheter assembly that may be used with the system shown in FIG. 1.

FIG. 3C is a perspective view of an alternative embodiment of a catheter assembly that may be used with the system shown in FIG. 1.

FIG. 3D is a perspective view of an alternative embodiment of a catheter assembly that may be used with the system shown in FIG. 1.

FIG. 3E is a perspective view of an alternative embodiment of a catheter assembly that may be used with the system shown in FIG. 1.

FIG. 4A is a perspective view of an alternative embodiment of a tip electrode array that may be used with the system shown in FIG. 1.

FIG. 4B is an exploded view of the tip electrode array shown in FIG. 4A.

FIG. 5 is a perspective view of an alternative embodiment of a catheter assembly that may be used with the system shown in FIG. 1.

FIG. 6A is a schematic diagram of one embodiment of a flex circuit architecture that may be used to implement the catheter assemblies described herein.

FIG. 6B is a schematic diagram of another embodiment of a flex circuit architecture that may be used to implement the catheter assemblies described herein.

FIG. 7 is a schematic diagram of an alternative embodiment of a catheter assembly that may be used with the system shown in FIG. 1.

FIGS. 8A and 8B are schematic diagrams of one embodiment of a mapping and ablation system.

FIG. 9 is a schematic diagram of one embodiment of a catheter assembly that may be used with the system shown in FIGS. 8A and 8B.

FIG. 10A is a perspective view of an alternative embodiment of a catheter assembly that may be used with the system shown in FIGS. 8A and 8B.

FIG. 10B is an end view of the catheter assembly shown in FIG. 10A.

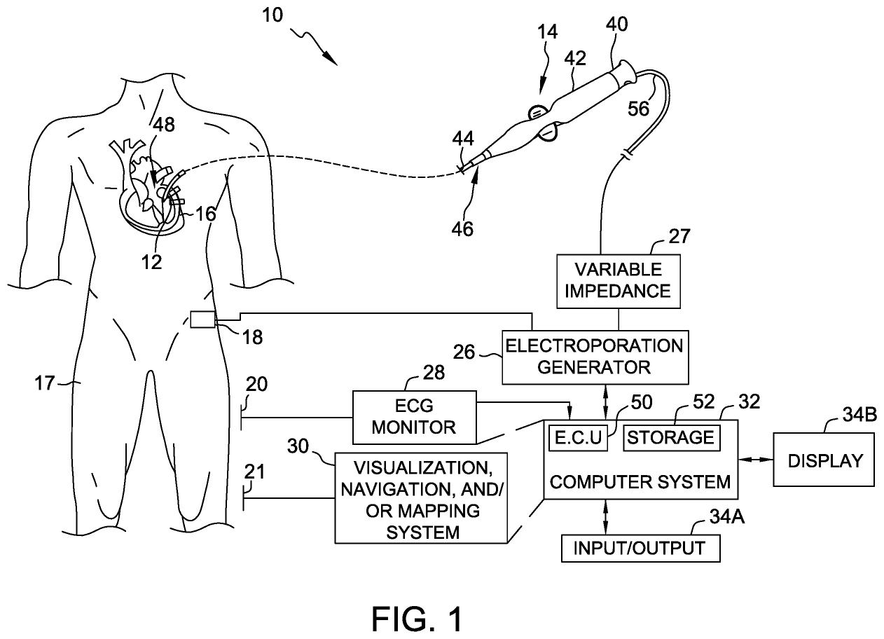

FIG. 7 is a schematic diagram of an alternative embodiment of a catheter assembly that may be used with the system shown in FIG. 1.

FIG. 11A is a side view of an alternative embodiment of a catheter assembly that may be used with the system shown in FIG. 1.

FIG. 11B is a perspective view of the tip electrode array of the catheter assembly shown in FIG. 11A.

FIGS. 12A and 12B are perspective view of an alternative embodiment of a catheter assembly that may be used with the system shown in FIG. 1.

FIG. 13 is a perspective view of an alternative embodiment of a catheter assembly that may be used with the system shown in FIG. 1.

### DETAILED DESCRIPTION OF THE DISCLOSURE

Systems and methods for a catheter assembly are provided. The catheter assembly includes a tip electrode array including at least one tip electrode, the tip electrode array located at a distal end of the catheter assembly, and a mini electrode array including a plurality of mini electrodes, the mini electrode array positioned proximal of the tip electrode array, wherein each of the at least one tip electrode and each of the plurality of mini electrodes are configured to be activated independent of one another for mapping applications, and wherein at least some of the at least one tip electrode and the plurality of mini electrodes are configured to be activated in unison for ablation applications.

Although at least some embodiments of the present disclosure are described with respect to pulmonary vein isolation (PVI), it is contemplated that the described features and methods of the present disclosure as described herein may be incorporated into any number of systems and any number of applications as would be appreciated by one of ordinary skill in the art based on the disclosure herein.

FIG. 1 is a block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

System 10 may be used for irreversible electroporation (IRE) to destroy tissue. In particular, system 10 may be used for electroporation-induced primary apoptosis therapy, which refers to the effects of delivering electrical current in such a manner as to directly cause an irreversible loss of plasma membrane (cell wall) integrity leading to its breakdown and cell apoptosis. This mechanism of cell death may be viewed as an “outside-in” process, meaning that the disruption of the outside wall of the cell causes detrimental effects to the inside of the cell. Typically, for classical plasma membrane electroporation, electric current is delivered as a pulsed electric field in the form of short-duration pulses (e.g., having a 0.1 to 20 millisecond (ms) duration) between closely spaced electrodes capable of delivering an electric field strength of about 0.1 to 1.0 kilovolts/centimeter (kV/cm). System 10 may be used, for example, for high output (e.g., high voltage and/or high current) electroporation procedures. In some particular embodiments, system 10 is configured to deliver an electroporation pulse signal having a relatively high voltage and low pulse duration.

In one embodiment, all electrodes of the catheter deliver an electric current simultaneously. Alternatively, in other embodiments, stimulation is delivered between pairs of electrodes on the catheter. Delivering electric current simultaneously using a plurality of electrodes may facilitate creating a sufficiently deep lesion for electroporation. To facilitate switching between i) activating electrodes simultaneously to deliver energy and ii) activating electrodes to sense signals (e.g., independent from one another), the electrodes may be switchable between being connected to a 3D mapping system and being connected to EP amplifiers.

It should be understood that while the energization strategies are described as involving DC pulses, embodiments may use variations and remain within the spirit and scope of the disclosure. For example, exponentially-decaying pulses, exponentially-increasing pulses, and combinations may be used. Further, in some embodiments, AC pulses may be used.

Further, it should be understood that the mechanism of cell destruction in electroporation is not primarily due to heating effects, but rather to cell membrane disruption through application of a high-voltage electric field. Thus, electroporation may avoid some possible thermal effects that may occur when using radio frequency (RF) energy. This “cold therapy” thus has desirable characteristics.

With this background, and now referring again to FIG. 1, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues.

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation-induced primary apoptosis therapy, generator 26 may be configured to produce an electric current that is delivered via electrode assembly 12 as a pulsed electric field in the form of short-duration DC pulses (e.g., a nanosecond to several milliseconds duration, a 0.1 to 20 ms duration, or any duration suitable for electroporation) between closely spaced electrodes capable of delivering an electric field strength (i.e., at the tissue site) of about 0.1 to 1.0 kV/cm. The amplitude and pulse duration needed for irreversible electroporation are inversely related. As pulse durations are decreased, the amplitude must be increased to achieve electroporation.

Electroporation generator 26, sometimes also referred to herein as a DC energy source, is a monophasic electroporation generator 26 configured to generate a series of DC energy pulses that all produce current in the same direction. In other embodiments, electroporation generator is biphasic or polyphasic electroporation generator configured to produce DC energy pulses that do not all produce current in the same direction. In some embodiments, electroporation generator 26 is configured to output energy in DC pulses at selectable energy levels, such as fifty joules, one hundred joules, two hundred joules, and the like. Other embodiments may have more or fewer energy settings and the values of the available setting may be the same or different. For successful electroporation, some embodiments utilize the two hundred joule output level. For example, electroporation generator 26 may output a DC pulse having a peak magnitude from about 300 Volts (V) to about 3,200 V at the two hundred joule output level. In some embodiments, the peak magnitude may be even larger (e.g., on the order of 10,000 V). Other embodiments may output any other suitable positive or negative voltage. For example, in some embodiments, the systems and methods described herein may include pulses with amplitudes from about 500 V to about 4,000 V, with pulse widths from about 200 nanoseconds to about 20 microseconds.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

In other embodiments, one or more semiconductor devices in series with catheter 14 may be used to limit arcing. For example, a specifically engineered semiconductor device, adapted from a field effect transistor, could be implemented, the device being a two terminal device capable of acting very quickly to limit current and power. Two of these devices may be used for biphasic energy delivery, while one device may be used for monophasic energy delivery. Commercially available devices are designed for low currents, usually in the milliamp range, but a semiconductor device for use in PFA applications could be engineered by modifying the size and/or dopant concentrations of existing devices. The would facilitate improving patient safety, and would potentially enable using catheters and generators multiple times.

With continued reference to FIG. 1, as noted above, catheter 14 may include functionality for electroporation and in certain embodiments also other types of ablation (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

In some embodiments, catheter 14 includes a basket catheter assembly having catheter electrodes (not shown in FIG. 1) distributed at the distal end of shaft 44 in a basket structure. Further, as described herein, an inflatable balloon may be contained within the basket structure.

Localization and navigation system 30 may be provided for visualization, mapping and navigation of internal body structures. Localization and navigation system 30 may include conventional apparatus known generally in the art (e.g., an EnSite Precision™ System, commercially available from Abbott Laboratories. and as generally shown with reference to commonly assigned U.S. Pat. No. 7,263,397 titled “Method and Apparatus for Catheter Navigation and Location and Mapping in the Heart,” the entire disclosure of which is incorporated herein by reference). It should be understood, however, that this system is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N.V., the AURORA® system of Northern Digital Inc., commonly available fluoroscopy systems, or a magnetic location system such as the gMPS system from Mediguide Ltd. In this regard, some of the localization, navigation and/or visualization system would involve a sensor be provided for producing signals indicative of catheter location information, and may include, for example one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

Pulsed field ablation (PFA) has been shown to be an effective form of ablation for treatment of cardiac arrhythmias, particularly for instantaneous pulmonary vein isolation (PVI). PFA includes delivering high voltage pulses from electrodes disposed on a catheter (e.g., including the basket and/or balloon catheters described herein). In PFA, for example, voltage amplitudes may range from about 300 V to at least 3,200 V (or even as large as on the order as 10,000 V), and pulse widths may from hundreds of nanoseconds to tens of milliseconds.

These electric fields may be applied between adjacent electrodes (in a bipolar approach) or between one or more electrodes and a return patch (in a monopolar approach).

Both approaches, using an appropriate electrode geometry and catheter placement, are able to provide contiguous lesions. For lesion size and proximity, the monopolar approach can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions.

To monitor operation of system 10, one or more impedances between catheter electrodes and/or return electrodes 18, 20, and 21 may be measured. For example, for system 10, impedances may be measured as described in U.S. Patent Application Publication No. 2019/0117113, filed on Oct. 23, 2018, U.S. Patent Application Publication No. 2019/0183378, filed on Dec. 19, 2018, and U.S. Patent Application No. 63/027,660, filed on May 20, 2020, all of which are incorporated by reference herein in their entirety.

The embodiments disclosed herein include catheters that are capable of performing both mapping and ablation functions. In general, the embodiments disclosed herein include catheters with an increased density of electrodes at the tip and distal portion (as compared to at least some known systems). This enables higher resolution, which in turn improves the ability of the system to define and locate arrhythmia features.

The embodiments described herein enable higher resolution mapping, generating higher fidelity electrograms (EGMs), and improving signal to noise (SNR). The higher resolution mapping results in improved mapping of borders/edges (e.g., the edges of scar tissue). These embodiments also provide improved techniques for detecting contact between a catheter and patient tissue. Further, the embodiments described herein provide omnipolar mapping technology (OT) in both two and three dimensions for catheter bodies. In addition, these embodiments may reduce the cost of fabricating catheter assemblies.

The catheter assemblies described herein include various arrangements of electrodes. The electrodes may operate independently from one another (in an “unganged” configuration) or may function together as a larger effective electrode (in a “ganged” configuration). For example, operating independent from one another, the electrodes may be used for mapping applications and/or EGM applications. In contrast, multiple electrodes may be tied together to function as a composite electrode for ablation applications and/or near field impedance navigation applications. Further, different subsets of electrode may be selectively activated (relative to one another) to provide improved control over ablation procedures.

As explained in detail below, the present disclosure provides catheter designs (e.g., linear, circular, basket) that allow for both cardiac mapping (in particular, OT) and ablation without requiring separate catheters for mapping and ablation or separate catheters for different ablation modalities.

Treating cardiac arrhythmias through selective ablation of cardiac tissue may be improved upon if, prior to ablation, the local electrical activity of the region can be mapped-especially when using OT and map visualization tools to confirm true presence of arrhythmia sources. This enables almost instantly treating the source of the arrhythmia without having to remove a diagnostic catheter and inserting an ablation catheter. This is particularly important because it is not generally feasible to locate a region or rhythm after removing a diagnostic catheter and then inserting an ablation catheter that may not support similar mapping technologies or electrode configurations.

At least some of the embodiments described herein provide a multi-modal ablation system and hybrid catheter designs with mini electrodes arranged in a an array (e.g., in a square array) that accommodates OT. In addition, the mini electrodes can serve to configure local tissue resistive load measurement. Such measurements provide improved assessment of contact between tissue and the catheter.

The ablation component enables delivering energy to and/or removing heat from the tissue. In at least some embodiments, the mapping component is capable of generating one or more different maps. An OT component uses, for example, the array of the mini electrodes on the hybrid catheter designs provided herein.

This disclosure also provides a processor that can selectively control the delivery of one or more different forms of treatment energy and selectively activate and control one or more energy treatment devices. Furthermore, methods of applying treatment energy to a target tissue area using multiple ablation techniques is also provided. For example, one method includes first providing a treatment energy generation station capable of supplying one or more different forms of treatment energy to one or more energy treatment devices, which are coupled to the treatment energy generation station. Treatment energy is then selectively supplied to one or more energy treatment devices. The target tissue area is ablated using one or more energy treatment devices.

Accordingly, hybrid catheter designs with mini electrodes that allow for PT and local impedance estimation are provided. Further, a multi-mode ablation system capable of delivering various energy types (such as cryoablation, microwave ablation, PFA, RF) is provided, as well as a system that allows for selective control and utilization of catheters to perform a variety of ablation strategies. While OT cardiac based mapping. RF ablation, PFA, microwave ablation, cryoablation and other ablation techniques are all useful, it becomes inconvenient, ineffective, and expensive to have to remove and replace existing catheters to effectively map and ablate a tissue region with a different form of ablation. Moreover, physicians have pointed out that when, say, a grid catheter (e.g., the Advisor™ HD Grid Mapping Catheter. or Sensor Enabled™ (SE) mapping catheter) that provides excellent electrogram properties and has OT capabilities is moved away from a site of interest, it may be very difficult to get the same view of signals or arrhythmias. As a result, it becomes difficult to perform ablation procedures as precisely as desired. The ablation system of this disclosure provides a unique design of catheters to allow for OT mapping and the ability to interact with existing catheters, of all designs, to effectively treat tissue treatment regions.

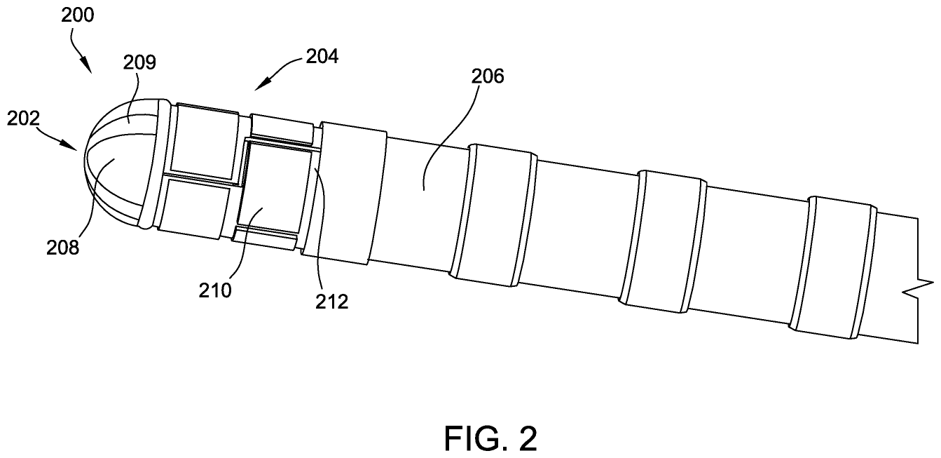

FIG. 2 is a perspective view of one embodiment of a catheter assembly 200 that may be used with system 10 (shown in FIG. 1). For example, catheter assembly 200 may be implemented as part of catheter 14. Catheter assembly 200 includes a tip electrode array 202, a mini electrode array 204, and a plurality of ring electrodes 206. In this embodiment, ring electrodes 206 are proximal of mini electrode array 204, and tip electrode array 202 is distal of mini electrode array 204.

As shown in FIG. 2, tip electrode array 202 includes a plurality of tip electrodes 208 separated by non-conductive segments 209. Specifically, tip electrode array 202 includes four tip electrodes 208 separated by four non-conductive segments 209. Tip electrode array 202 also has a dome shape.

In the embodiment of FIG. 2, mini electrode array 204 includes a plurality of rectangular electrodes 210 wrapped around a circumference of catheter assembly 200. Further, rectangular electrodes 210 are arranged in a brick-like pattern on a non-conductive substrate 212 that insulates rectangular electrodes 210 from one another.

For mapping applications, tip electrodes 208 and rectangular electrodes 210 may function or be activated independent from one another (i.e., may sense voltages independent from one another, be energized independent from one another, may be energized at different polarities from one another, and/or may be energized at different voltages from one another). For example, using tip electrodes 208 and rectangular electrodes 210 to sense voltages independent from one another facilitates obtaining navigation impedance information that may be used to accurately display the catheter and generating independent electrograms.

In contrast, for ablation applications, two or more of tip electrodes 208 and rectangular electrodes 204 are activated in unison to form a larger effective electrode. For example, if all rectangular electrodes 204 are activated in unison, they effectively function as a ring electrode (similar to ring electrodes 206). Those of skill in the art will appreciate that any suitable combination of electrodes may be activated in unison.

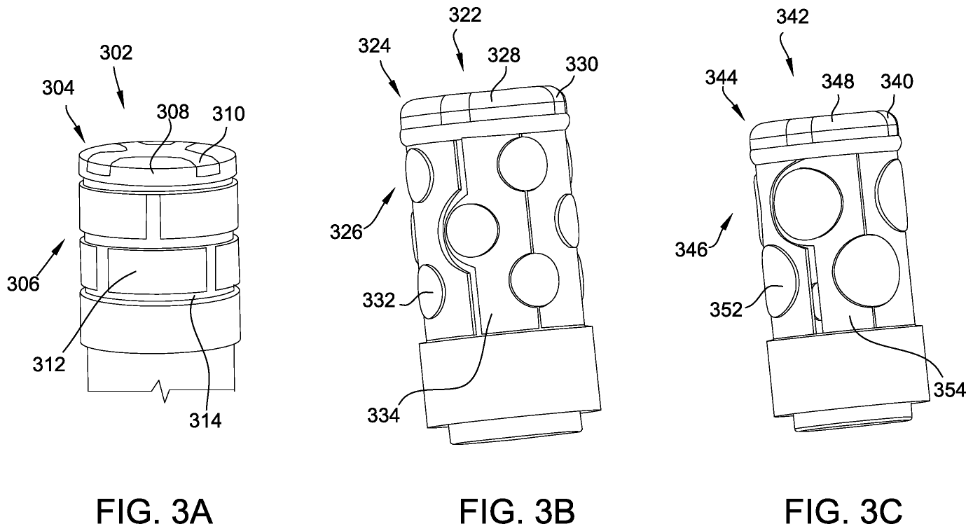

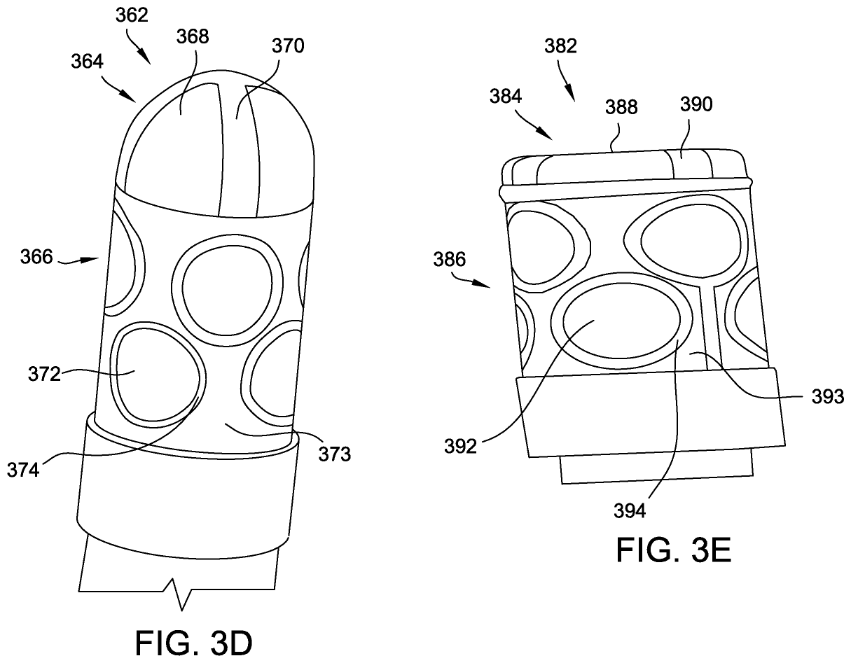

FIG. 2 is one example of a catheter assembly that includes a tip electrode array and a mini electrode array. Those of skill in the art will appreciate that many other configurations are possible. For example, FIGS. 3A-3E are perspective view of alternative catheter assemblies.

FIG. 3A shows a catheter assembly 302 that includes a tip electrode array 304 and a mini electrode array 306. Tip electrode array 304 includes a plurality of tip electrodes 308 separated by non-conductive segments 310. Here, tip electrode array 304 also has a flat shape (as opposed to the domed shaft of tip electrode array 202 (shown in FIG. 2)).

In the embodiment of FIG. 3A, mini electrode array 306 includes a plurality of rectangular electrodes 312 wrapped around a circumference of catheter assembly 302. Further, rectangular electrodes 312 are arranged in a brick-like pattern on a non-conductive substrate 314 that insulates rectangular electrodes 312 from one another.

FIG. 3B shows a catheter assembly 322 that includes a tip electrode array 324 and a mini electrode array 326. Tip electrode array 324 includes a plurality of tip electrodes 328 separated by non-conductive segments 330. Here, tip electrode array 324 has a flat shape.

In the embodiment of FIG. 3B, mini electrode array 326 includes a plurality of circular electrodes 332 arranged around a circumference of catheter assembly 322. Further, circular electrodes 332 are arranged in a spaced apart pattern on a non-conductive substrate 334 that insulates circular electrodes 332 from one another.

FIG. 3C shows a catheter assembly 342 that includes a tip electrode array 344 and a mini electrode array 346. Tip electrode array 344 includes a plurality of tip electrodes 348 separated by non-conductive segments 350. Here, tip electrode array 344 has a flat shape.

In the embodiment of FIG. 3C, mini electrode array 346 includes a plurality of circular electrodes 352 arranged around a circumference of catheter assembly 342. Further, circular electrodes 352 are arranged in a spaced apart pattern on a non-conductive substrate 354 that insulates circular electrodes 352 from one another.

FIG. 3D shows a catheter assembly 362 that includes a tip electrode array 364 and a mini electrode array 366. Tip electrode array 364 includes a plurality of tip electrodes 368 separated by non-conductive segments 370. Here, tip electrode array 364 has a dome shape.

In the embodiment of FIG. 3D, mini electrode array 366 includes a plurality of circular electrodes 372 arranged around a circumference of catheter assembly 362. Further, circular electrodes 372 are arranged in a spaced apart pattern. In this embodiment, an annular electrode 373 extends around catheter assembly 362, and circular electrodes 372 are positioned within apertures defined by annular electrode 373. A non-conductive substrate 374 insulates circular electrodes 372 from one another and from annular electrode 373. During an ablation application, circular electrodes 372 and annular electrode 373 may be activated in unison to function as a ring electrode.

FIG. 3E shows a catheter assembly 382 that includes a tip electrode array 384 and a mini electrode array 386. Tip electrode array 384 includes a plurality of tip electrodes 388 separated by non-conductive segments 390. Here, tip electrode array 384 has a flat shape.

In the embodiment of FIG. 3E, mini electrode array 386 includes a plurality of circular electrodes 392 arranged around a circumference of catheter assembly 382. Further, circular electrodes 392 are arranged in a spaced apart pattern. In this embodiment, an annular electrode 393 extends around catheter assembly 382, and circular electrodes 392 are positioned within apertures defined by annular electrode 393. A non-conductive substrate 394 insulates circular electrodes 392 from one another and from annular electrode 393. During an ablation application, circular electrodes 392 and annular electrode 393 may be activated in unison to function as a ring electrode.

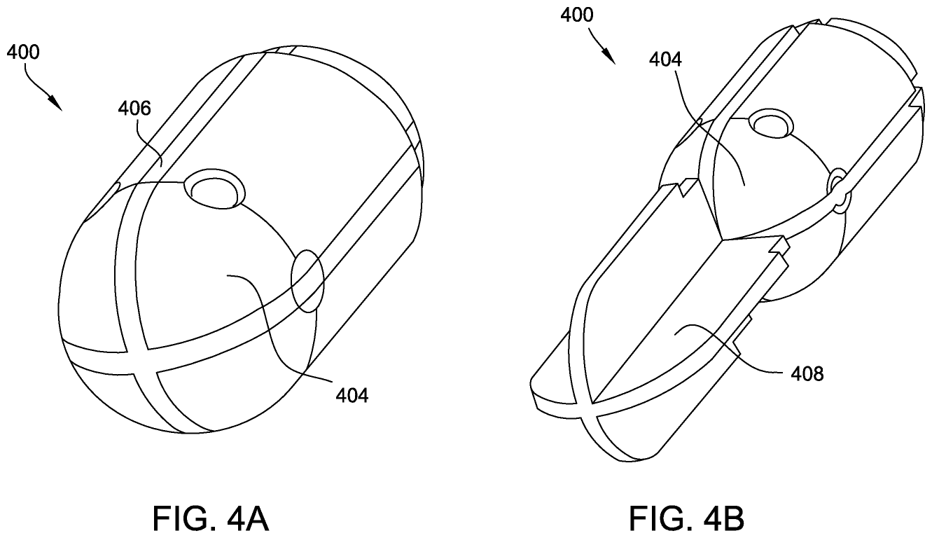

FIG. 4A is perspective view of another embodiment of a tip electrode array 400, and FIG. 4B is an exploded view of tip electrode array 400. Tip electrode array 400 includes a plurality of tip electrodes 404 separated by non-conductive segments 406. Specifically, tip electrode array 400 includes four tip electrodes 404 separated by four non-conductive segments 406. Tip electrode array 400 also has a dome shape. As shown best in FIG. 4B, in this embodiment, non-conductive segments 406 are formed by a single non-conductive insert 408 that is inserted between the four tip electrodes 404.

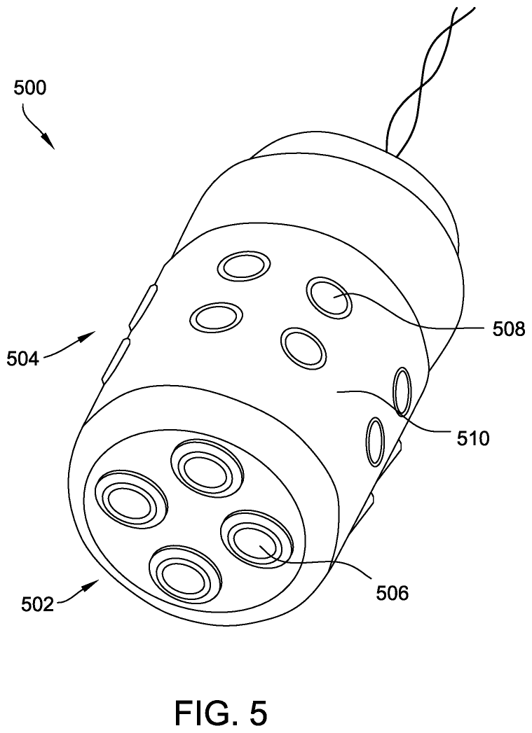

FIG. 5 is a perspective view of another embodiment of a catheter assembly 500. Catheter assembly 500 includes a tip electrode array 502 and a mini electrode array 504. Tip electrode array 502 includes a plurality of circular tip electrodes 506 arranged in a grid. Further, mini electrode array 504 includes a plurality of circular electrodes 508 arranged in a grid about a circumference of catheter assembly 500. Circular tip electrodes 506 and circular electrodes 508 are positioned in apertures defined within a substrate 510. In some embodiments, the substrate 510 is conductive (and may be activated in unison with circular tip electrodes 506 and/or circular electrodes 508 for ablation applications), while in other embodiments, the substrate 510 is non-conductive.

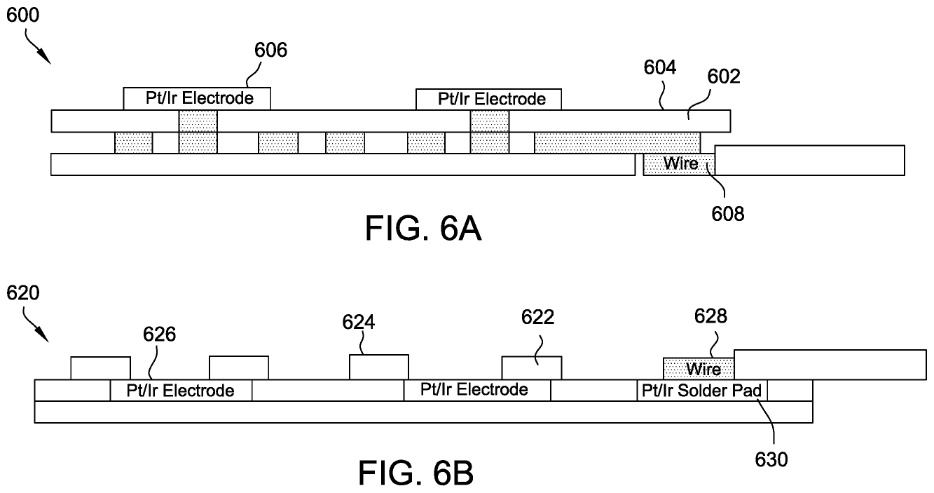

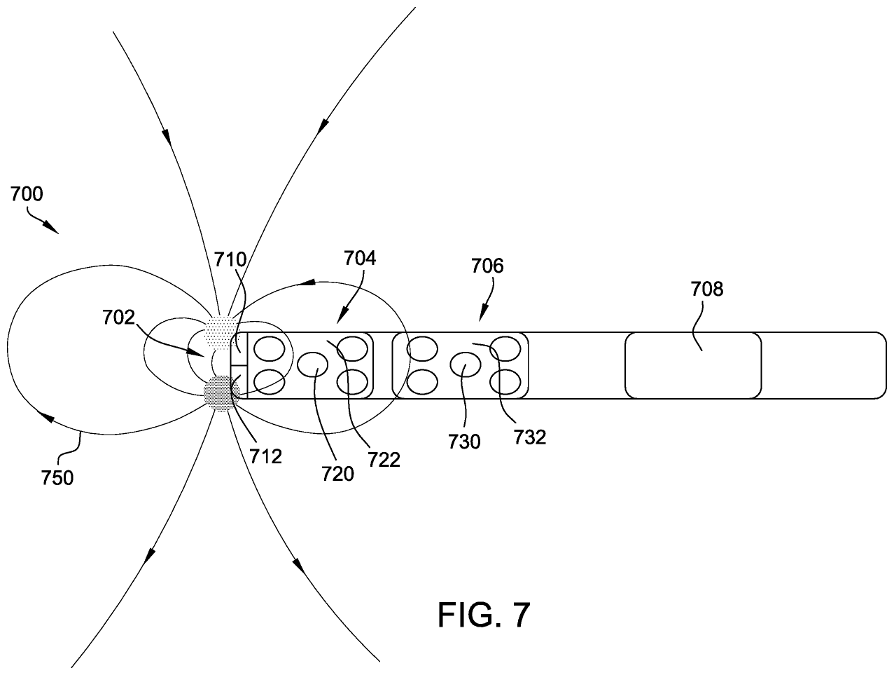

FIG. 6A is a schematic diagram of one embodiment of a flex circuit architecture 600 that may be used to implement the catheter assemblies described herein. Flex circuit architecture 600 may be used, for example to implement the mini electrode arrays shown in FIGS. 2-5. Those of skill in the art will appreciate that flex circuit architecture 600 could be wrapped into an annular shape to form those mini electrode arrays.

Flex circuit architecture 600 includes a substrate 602 with an outer surface 604. A plurality of electrodes 606 (e.g., platinum/iridium (Pt/Ir) electrodes) are positioned on the outer surface 604. Wiring 608 for supplying energy to electrodes 606 extends through substrate 602 and connects to a back side of electrodes 606.

FIG. 6B is a schematic diagram of an alternative embodiment of a flex circuit architecture 620 that may be used to implement the catheter assemblies described herein. Flex circuit architecture 620 may be used, for example to implement the mini electrode arrays shown in FIGS. 2-5. Those of skill in the art will appreciate that flex circuit architecture 620 could be wrapped into an annular shape to form those mini electrode arrays.

Flex circuit architecture 620 includes a substrate 622 with an outer surface 624. Here, in contrast to flex circuit architecture 600, a plurality of electrodes 626 are embedded below outer surface 624. Further, wiring 628 for supplying energy to electrodes 626 connects to an outer surface of a solder pad 630 (e.g., a Pt/Ir pad), and solder pad 630 is in turn electrically connected to one or more electrodes 626.

FIG. 7 is a schematic diagram of another embodiment of a catheter assembly 700. Catheter assembly 700 includes a tip electrode array 702, a first mini electrode array 704, a second mini electrode array 706, and a ring electrode 708. Tip electrode array 702 includes a first tip electrode 710 and a second tip electrode 712. First mini electrode array 704 includes a plurality of circular electrodes 720 and a conductive substrate 722, and second mini electrode array includes a plurality of circular electrodes 730 and a non-conductive substrate 732. Those of skill in the art will appreciate that catheter assembly 700 includes an example configuration of electrodes, and that other arrangements of electrodes are within the spirit and scope of the disclosure.

FIG. 7 illustrates electric field lines 750 representing an electric field generated when first and second tip electrodes 710 and 712 are used in a bipolar configuration (i.e., with a voltage generated between first and second tip electrodes 710 and 712). Those of skill in the art will appreciate that any suitable pair of electrodes from among tip electrode array 702, first mini electrode array 704, second mini electrode array 706, and/or ring electrode 708 may be used in a bipolar configuration.

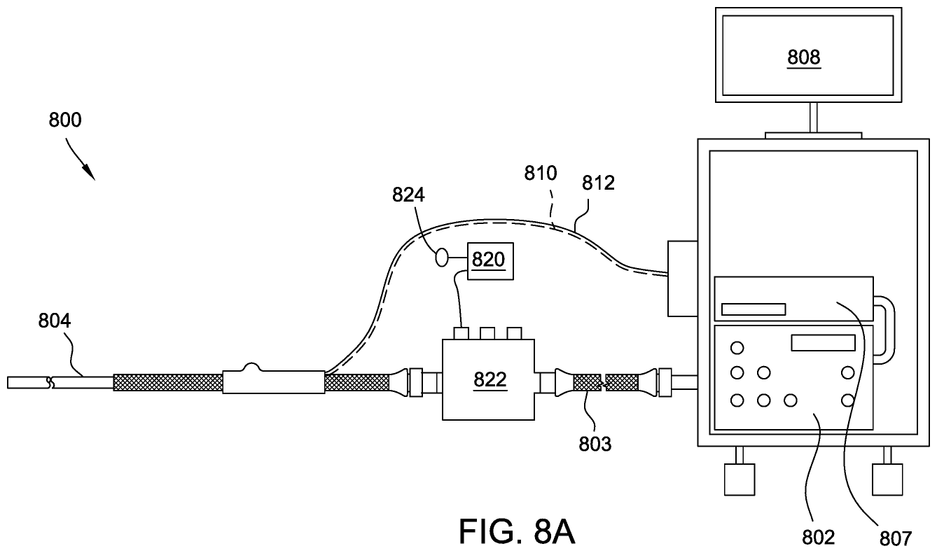

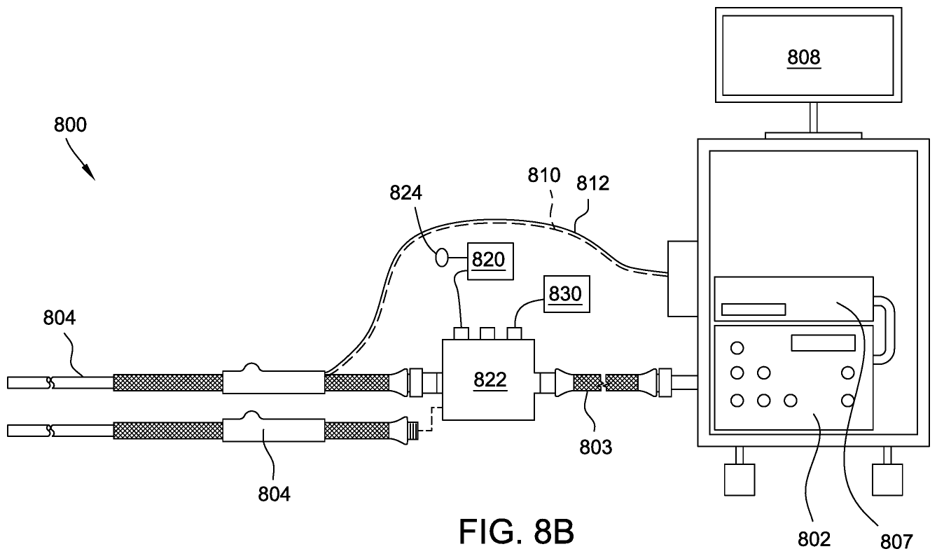

FIG. 8A is a schematic diagram of one embodiment of a mapping and ablation system 800. System 800 is capable of performing both RF ablation and PFA, as well as other ablation types (e.g., microwave ablation and cryoablation). System 800 includes a console 802, a catheter 804, and a cable system 803 coupling console 802 to catheter 804.

Catheter 804 may be, for example, a linear catheter, a loop catheter, and/or any other suitable energy treatment device that may easily and smoothly move through blood vessels and heart valves. Cable system 803 includes electrical signal lines for monitoring and/or mapping tissue and cardiac regions, and may be coupled to a mapping system 807 and an ECG/electrogram monitor 808. Cable system 803, in this embodiment, includes a cooling injection cable system 810 and a vacuum cable system 812 that provide respective inlet and return paths for a coolant used to cool a tissue-treating end of catheter 804.

Console 802 provides a user interface for system 800, and houses electronics and software for controlling and recording mapping and ablation procedures, controlling the delivery of the liquid coolant under pressure through cable system 803 to catheter 804, controlling recovery of an expanded refrigerant vapor from catheter 804 under vacuum, and controlling a compressor to pressurize the coolant vapor into a liquid stored in a recovery tank. In addition to the liquid coolant, a secondary heat removal or dissipation element, such as a conductive coil, may be used.

System 800 produces controlled temperatures and or pulses at a tip of catheter 804. One or more selected catheters 804 may be coupled to console 802. For example, FIG. 8B shows system 800 with two catheters 804 connected. Generally, catheters 804 are sufficiently long and flexible to be inserted through various body conduits. System 800 may be used with catheters designed for both endovascular and open surgical procedures. System 800 is configured to supply more than one type of energy to catheters 800 (e.g., system 800 may be used with catheters 804 that are adaptable for PFA, RF ablation, cold-tipped RF ablation, microwave ablation, cryoablation, and/or mapping procedures—specifically OT). At least one of catheters 804 may be a focal-tip catheter that produces a concentrated zone of tissue destruction (i.e., ablation), a linear catheter that delivers cold along the length of the catheter, or a loop catheter suitable for PVI isolation.

In the embodiment shown in FIGS. 8A and 8B, a radiofrequency generator 820 is coupled to a generator hub 822. Using controls on console 2, a user may select the type of ablation procedure he or she wishes to perform.

Catheter 804 may include one or more electrodes around its periphery. If the user wishes to perform an RF ablation procedure, radiofrequency energy can be provided to the electrodes of catheter 804 via cable system 803 to perform an RF ablation technique as is common in the cardiac ablation state of the art. Specifically. RF energy is provided between electrodes situated on or within catheter 804 and an indifferent electrode 824 (also known as a collector plate). Indifferent electrode 824 may be positioned, for example, on the patient's body. RF energy flows within the patient's tissue between the electrodes on catheter 804 and indifferent electrode 824, causing cell death of or treating the targeted tissue.

In some embodiments, a conductive coolant, by itself or concurrent with RF current delivery, may be delivered to catheter 804 to complete the electrical connection to the electrodes. Direct current (DC) may also be supplied to catheter 804.

The mechanism underlying lesion formation from DC shocks is known in the art of electrophysiology, and results in irreversible electroporation IRE. Lesion formation in IRE is a function of electric field exposures that break down cell membrane permeability and homeostasis. This leads to cell death, while at the same time preserving the integrity and function of nearby structures, such as the esophagus, lungs, coronary arteries, PVs, and phrenic nerve.

As described above, PFA is a form of IRE that uses a train of monophasic, or biphasic pulses of high voltage and short duration to cause cell death in tissue without significant heating (when waveform parameters are optimized appropriately). Tissue effects at a target location are directly controlled by the magnitude and duration of the applied electric field. Voltage directly affects the electric field intensity distribution; that is, an increase in voltage will result in an increase in therapy strength. When voltage magnitudes increase, tissue heating increases, muscles may contract, and a volume of gaseous microemboli (i.e., microbubbles) may be generated.

For PFA, when multiple pulses are delivered in rapid succession (e.g., over nanosecond to millisecond timeframes), their effects may be viewed as a collective pulse, referred to as a packet. When multiple packets are delivered to tissue, the accumulated injury to the cell increases. Thus, more packets will result in stronger treatment effects but will increase the treatment delivery time (for a matched packet delivery rate) and will increase the cumulative temperature rise if there is insufficient time for heat dissipation between subsequent packets. When a pause is provided between a sequence of pulses or a packet, the tissue gets an opportunity to conduct heat away from the warmed-up tissues and therefore reduce total temperature rise. When a sequence of pulses is delivered with a relatively larger pause (milliseconds to tens of seconds), cellular recovery is generally unaffected, but the total tissue temperature rise is reduced. This enables delivery of strong cumulative treatments while maintaining acceptable levels of temperature rise.

As shown in FIG. 8B, in addition to RF generator 820, a PFA generator 830 may also be coupled to hub 822. The PFA energy may also be delivered to patient through catheter 804. Those of skill in the art will appreciate that other energy sources (e.g., a cryo-ablation generator) may be selectively coupled to hub 822 in other embodiments.

Accordingly, system 800 allows for various combinations of different types of ablation energy to be delivered to one or more catheters 804, which ablate the target tissue region. The ablation procedures can take place in a sequential manner, depending upon the desired therapy strategy for treating the tissue. The possibility of quickly switching from one ablation procedure to another may lead to better tissue lesion treatment outcomes.

For example, in one scenario, where a physician desires to achieve a deep lesion, the tissue may first be cryo-ablated for 45 seconds, until a local edema is created. Then RF ablation may follow, after extracellular fluid has accumulated proximate the tissue region to allow RF energy to more easily spread deeper into the tissue region.

In another example, a target tissue region near the esophagus is mapped in combination with OT. For this purpose, a shallow lesion of say 1-3 mm depth is sufficient to complete an isolation. PFA alone is then used, with a user-set field strength of about 300V/cm.

In yet another example, suppose a physician desires to administer PFA in a confined zone. Cryoablation may be performed first. Notably, when PFA is subsequently applied in low temperature ice created by the cryoablation, it is confined to the ice. This allows for the possibility of direct visualization of the ablation zone with traditional imaging technologies.

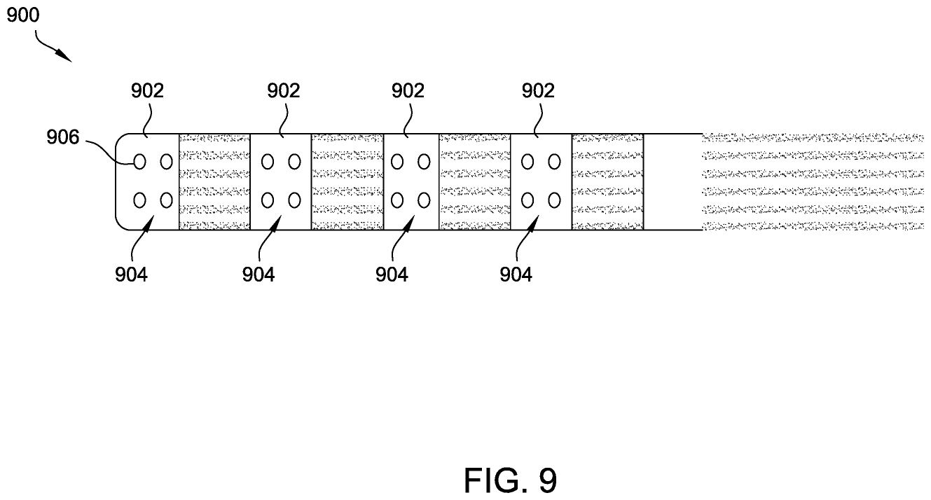

FIG. 9 is a schematic diagram of one embodiment of a catheter assembly 900 that may be used with system 800 (shown in FIGS. 8A and 8B) or with system 10 (shown in FIG. 1). Catheter assembly 900 is a linear assembly that includes a plurality of ring electrodes 902. Further, for at least some of ring electrodes 902, a mini electrode array 904 is formed around the ring electrode 902. In this embodiment, each mini electrode array 904 includes four mini electrodes 906 arranged in a square grid pattern, with mini electrodes 906 evenly spaced from one another. Although one mini electrode array 904 is shown on each ring electrode 902, in some embodiments, a single ring electrode 904 may include more than one mini electrode array 902 thereon.

Mini electrode arrays 904 may be used to tailor application of electrical energy (e.g., to generate omnipolar patterns and/or directed applications of pacing, RF, or PFA energy). Further, mini electrode arrays 904 take advantage of OT and enable sensing consistent voltage signals regardless of orientation and detecting wave propagation speeds and direction. Specifically, the square arrangement of mini electrode arrays 904 supports OT mapping technology, and the mini electrode arrays 904 may be used in multiple catheter designs (e.g., linear or loop catheters). Using mapping systems, a grid arrangement of mini electrode arrays 904 enables displaying directional properties, propagation speed, and/or maximal voltage electrograms, regardless of catheter orientation. Further, it is feasible to measure an impedance load locally on the tissue-catheter interface using mini electrode arrays 904, thus providing improved discrimination between the myocardium and the blood pool. The impedance load on the distal electrode of an ablation catheter is largely influenced by the ratio of surface area covered by myocardium and blood. Myocardium impedance is larger than that of blood pool (e.g., 3.0-6.0 Ohm-meters (Ω-m) and 1.5 Ω-m, respectively). Notably, increased resistivity of the myocardium is responsible for its preferential Joule heating when compared to blood

Several technologies have attempted to measure the resistive load at the catheter-tissue interface via impedance. Traditionally, RF generators estimate resistive loads via transthoracic impedance of the energy delivery pathway from an ablation catheter tip electrode to an indifferent electrode on the skin. While RF generators can reasonably estimate impedance differences between tissue and blood, they are hindered by large variations of bulk impedance of the torso such as muscles, lung, and bone.

In contrast, using the embodiments described herein, resistive loads can be measured more locally by taking advantage of mini electrode arrays 904 incorporated into larger ring electrodes 902. For example, non-stimulating AC current may be driven between the distal and proximal electrodes of a linear ablation catheter, creating a local potential field. Mini electrodes 906 can then be used to measure variations in the potential field that may be due to nearby cardiac tissue. The measured potential can be converted into impedance by dividing by an injection current.

It is known that catheter impedance plateaus before reaching unsafe contact forces. Accordingly, local impedance measurement on the ablation catheter can serve as a surrogate for the distal electrode surface area covered by myocardium. Although contact force may provide safety benefits, a force value is unreliable predictor of catheter-tissue surface area coverage and resistive heating during radiofrequency applications. For example, a catheter that is lightly placed in trabeculae may have low contact force but still can deliver significant radiofrequency energy to the myocardium because the distal electrode is covered by tissue.

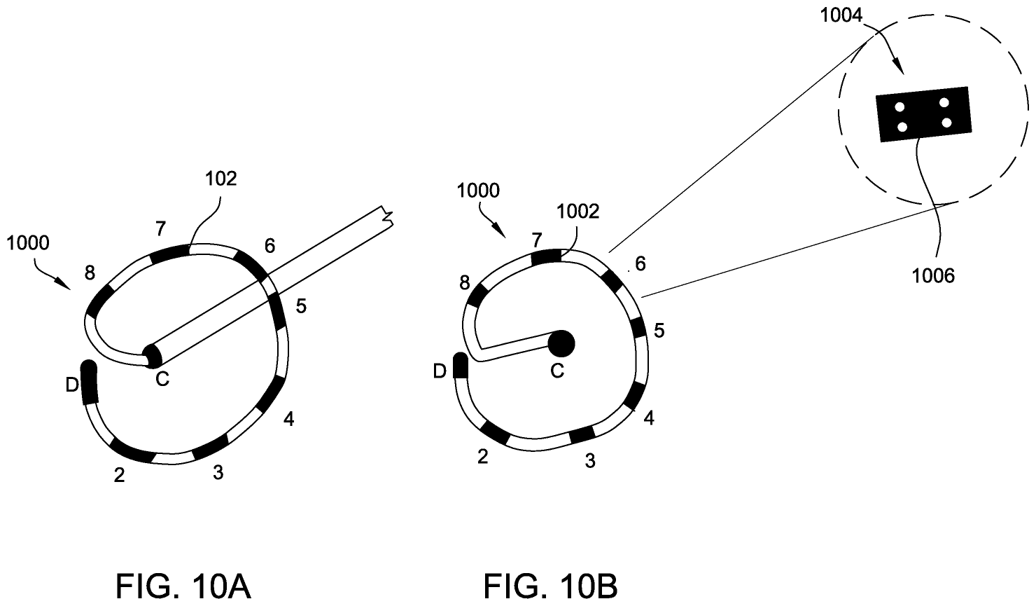

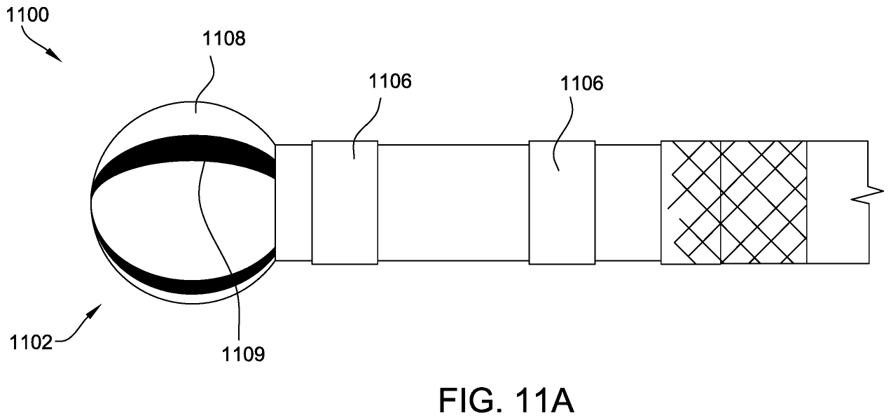

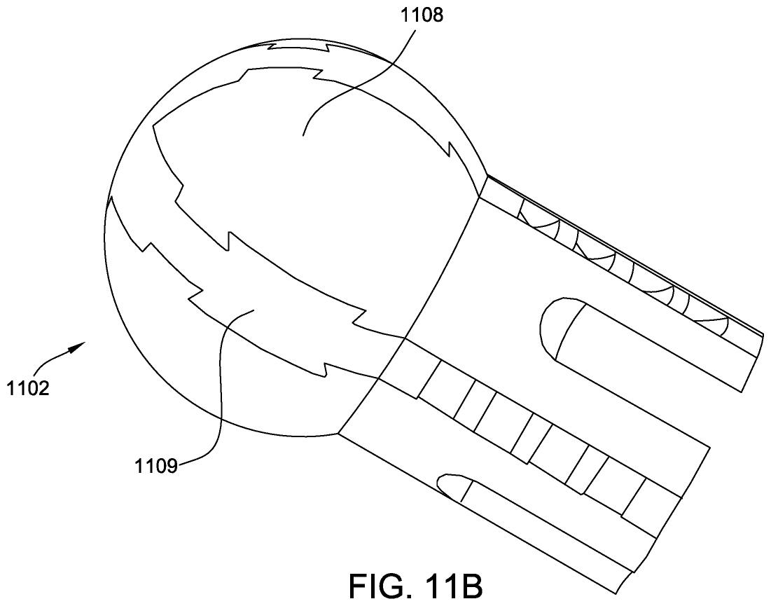

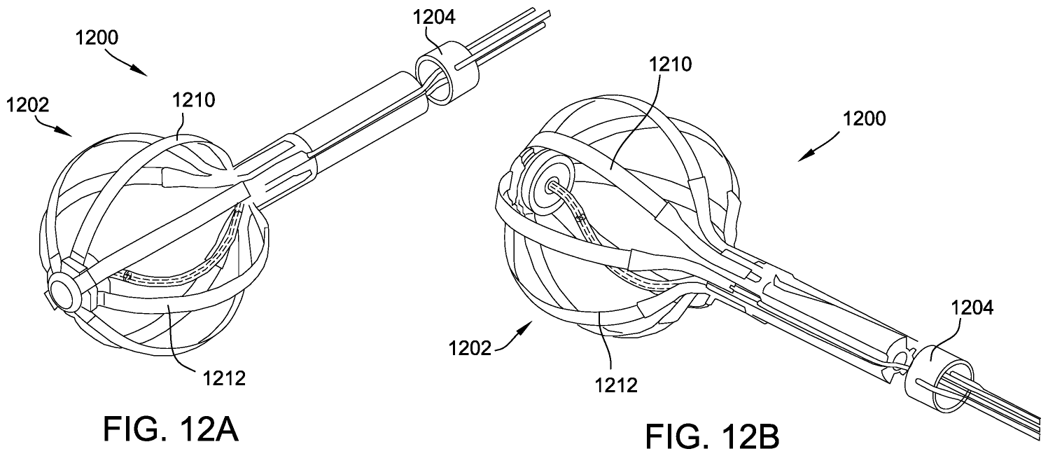

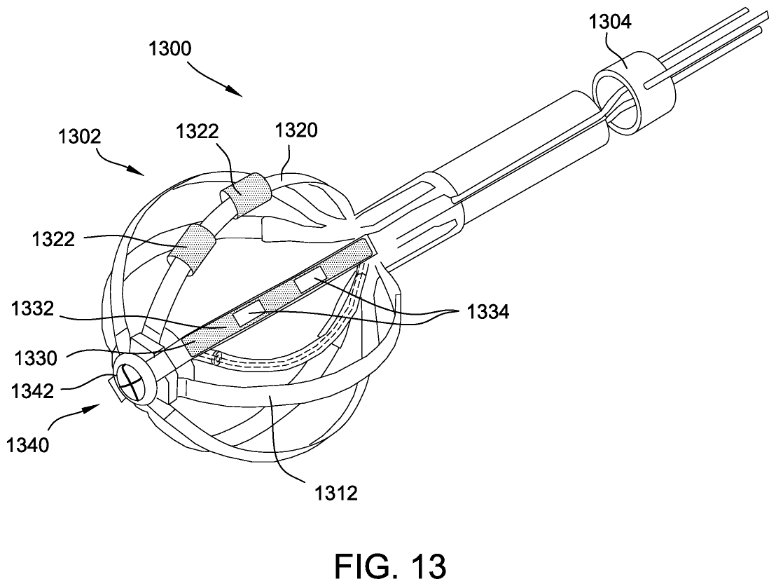

FIG. 10A is a perspective view of one embodiment of a catheter assembly 1000 that may be used with system 800 (shown in FIGS. 8A and 8B). FIG. 10B is an end view of catheter assembly 1000. As shown in FIGS. 10A and 10B, catheter assembly 1000 is a loop assembly. Catheter assembly 1000 includes a plurality of ring electrodes 1002. Further, for at least some of ring electrodes 1002, a mini electrode array 1004 is formed on the ring electrode 1002 (see enlarged portion). In this embodiment, each mini electrode array 1004 includes four mini electrodes 1006 arranged in a square grid pattern, with mini electrodes 1006 evenly spaced from one another. Although one mini electrode array 1004 is shown on each ring electrode 1002, in some embodiments, a single ring electrode 1004 may include more than one mini electrode array 1002 thereon. Mini electrode arrays 1004 function substantially similar to mini electrode arrays 904 (shown in FIG. 9).

Catheter assembly 1000 includes nine ring electrodes 1002 in this embodiment (e.g., a central electrode (“C”) and eight outer electrodes (“D”, “2”, “3”, “4”, “5”, “6”, “7”, and “8”) adjacent to or surrounding the central electrode). The central electrode may or may not lie in the same plane as the outer electrodes. Further, in some embodiments, catheter assembly 100 includes two central electrodes: one in the same plane as the outer electrodes, and one outside of that plane.

Alternatively, catheter assembly 1000 may include any suitable number of ring electrodes 1002. Catheter assembly 1000 may be useful for pulmonary vein ablation. It can be powered by a duty-cycled radiofrequency (RF) generator or a specially designed PFA generator. Catheter assembly 100 may efficiently create contiguous, transmural lesions capable of PV isolation. In addition, pulsed electric field energy delivery through catheter assembly 1000 is possible, and can create contiguous transmural myocardial lesions like RF energy. The PFA energy design can be configured to deliver high-voltage, bipolar pulse trains to catheter assembly 1000 through a cable that connects electrodes 1002 “D”, “3”, “5”, and “7” as one polarity, and electrodes 1002 “2”, “4”, “6”, and “8” as the opposite polarity. Duty-cycled RF energy can be delivered in a 2:1 bipolar/unipolar ratio, a maximum power of 10 Watts (W) per electrode, and 60° C. temperature setpoint for 60 seconds at each placement. PF energy can be delivered as biphasic pulse trains with a pulse width of 100 microseconds (μs) for each phase and with 200 us inter pulse pauses. Those of skill in the art will appreciate that these parameters are merely an example of possible parameters.

In an electroanatomic mapping scenario, a user, via a menu on monitor 808 (shown in FIG. 8) applies a set of mapping configurations. Specifically, monitor 808 may be used to select various map types for investigating electrograms of interest on the display. Because the spacing between mini electrodes is relatively small, generally only local information will be provided to a physician. A loop catheter, however, such as catheter assembly 1000, can be configured to provide both local and a larger view of the target region.

Referring back to FIGS. 8A and 8B, catheters 804 may be any suitable RF ablation, PFA, microwave ablation, and/or cryoablation catheters, as well as hybrid catheters with the ability to ablate tissue via combinations of PFA, RF, microwave ablation, and cryoablation. A computing device (e.g., included in console 802) allows a user to selectively control which catheter 804 will receive a chosen energy type. A user may, for example, choose to treat target tissue with a traditional RF procedure. Further, mapping may be done before an ablation procedure.

Alternatively, the user may choose to treat the target tissue using a PFA procedure. The computing device is then directed to access a PF-capable catheter 804 already coupled to the system via cable system 3. Prior to the lesions being created by the PFA, electroanatomic mapping may be performed first. This may be accomplished by first enabling a desired catheter 804 for mapping the desired area. Any combination of ablation procedures can treat the target tissue by first enabling a compatible cryo-treatment catheter, microwave catheter, RF ablation catheter, or PFA catheter. Further, the same catheter can be used to follow ablation immediately by remapping to assess the effects of therapy applications. In this fashion, multiple maps and a wide range of temperature ranges can be achieved without the need to change and/or replace one type of catheter 804 with another.

Using the embodiments described herein, any desired combination of ablation procedures may be performed on a target tissue area. For example, ablation may be performed with or without a mapping procedure preceding it. Further, any suitable sequence of ablation procedures may be performed. For example, RF ablation followed by PFA may be performed, especially in areas near circulatory “heat sinks” such as blood vessels known to limit lesion formation with traditional ablation techniques.

A multi-mode procedure may also be implemented wherein a sequence of ablation procedures is programmed and automatically controlled by the computing device. Specifically, the user provides input to the computing device, and these inputs result in activation signals to appropriate catheters 804 to control the delivered energy type, as well as ancillary functionality such as circulating cooling fluid to catheters 804.

Simultaneous RF ablation, PFA, microwave ablation, and/or cryoablation is also possible using the embodiments described herein. That is, different types and quantities of ablation energies may be delivered to catheters 804 to perform simultaneous ablation procedures. For example, in a multi-catheter scenario, one catheter 804 may receive coolant for a cryoablation procedure, while RF energy may be supplied to another catheter 804 via the RF generator 820, and PFA energy may be supplied to yet another catheter 804. Further, microwave energy may be applied to yet another catheter 804.

The embodiments described herein are not limited to a specific number of catheters, or a specific sequence of ablation sequences. The multi-energy ablation systems described herein allows for a variety of different types of probes or catheters, each enabled to perform one or more ablation procedures, coupled to a control system that selectively supplies various types of energy necessary for mapping and ablating an area of the body. Such systems would be very helpful to practicing physicians in cardiac electrophysiology, especially because these systems may overcome shortcomings of various ablation modalities in at least some known systems.

FIG. 11A is a side view of another embodiment of a catheter assembly 1100 that may be used with the systems and methods described herein (e.g., system 10, shown in FIG. 1, or system 800, shown in Figures &A and 8B). For example, catheter assembly 1100 may be implemented as part of catheter 14. Catheter assembly 1100 includes a tip electrode array 1102 and a plurality of ring electrodes 1106. FIG. 11B is a perspective view of tip electrode array 1102.

As shown in FIGS. 11A and 11B, tip electrode array 1102 includes a plurality of tip electrodes 1108 separated by non-conductive segments 1109. Specifically, tip electrode array 1102 includes four tip electrodes 1108 separated by four non-conductive segments 1109. Tip electrode array 1102 also has a generally spherical shape. Non-conductive segments 1109 may be formed by a single non-conductive insert, similar to non-conductive insert 408 (shown in FIG. 4B).

The shape of tip electrode array 1102 enables catheter assembly 1100 to generate relatively uniform lesions regardless of a contact angle between the catheter assembly 1100 and patient tissue. That is, using catheter assembly 1100 prevents or significantly reduces the generation of shadow lesions (e.g., lesions inadvertently generated during therapy). The tip electrode array 1102 may have anu suitable size. For example, in some embodiments, the spherical portion of tip electrode array 1102 has a diameter of approximately 4.19 mm, 3.94 mm, or 2.34 mm.

The relatively large diameter of tip electrode array 1102 may have other advantages as well. For example, the larger diameter will necessarily decrease the rate at which a local field strength decreases, suggesting more uniform RF and PFA effect radii.

FIG. 12A is a perspective view of another embodiment of a catheter assembly 1200 that may be used with the systems and methods described herein (e.g., system 10, shown in FIG. 1). For example, catheter assembly 1200 may be implemented as part of catheter 14. Catheter assembly 1200 includes a tip electrode array 1202 and a ring electrode 1204. FIG. 12B is another perspective view of catheter assembly 1200.

As shown in FIGS. 12A and 12B, tip electrode array 1202 includes a basket 1210 with a plurality of conductive splines 1212 that serve as electrodes. Basket 1210 may have a diameter, for example, of approximately 1 centimeter (cm). In some embodiments, a pull member (e.g., a pull ring and/or pull wire) may be used to selectively increase the diameter of basket 1210 (e.g., by approximately 0.152 mm). Splines 1212 may function as independent electrodes from one another, or may be activated in unison. Like catheter assembly 1100 (shown in FIGS. 11A and 11B), the shape of tip electrode array 1202 enables catheter assembly 1200 to generate relatively uniform lesions regardless of a contact angle between catheter assembly 1200 and patient tissue. Further, applying energy between a tip electrode (e.g., electrode 1108 in FIG. 11A) and a shaft electrode closest to the tip electrode (e.g., the leftmost ring electrode 1106 in FIG. 11A) also facilitates reducing the effect of the contact angle on lesion shape.

In the embodiment of FIGS. 12A and 12B, each spline 1212 includes a single electrode. Alternatively, one or more splines in a basket catheter assembly may include a plurality of electrodes. For example, FIG. 13 is a perspective view of another embodiment of a catheter assembly 1300 that may be used with the system and methods described herein (e.g., system 10, shown in FIG. 1). Catheter assembly 1300 includes a tip electrode array 1302 and a ring electrode 1304.

As shown in FIG. 13, tip electrode array 1302 includes a basket 1310 with a plurality of splines 1312 that each include one or more electrodes. Basket 1310 may have a diameter, for example, of approximately 1 centimeter (cm). In some embodiments, a pull member (e.g., a pull ring and/or pull wire) may be used to selectively increase the diameter of basket 1310 (e.g., by approximately 0.152 mm).

In this embodiment, a first spline 1320 includes multiple ring electrodes 1322. Further, a second spline 1330 includes an elongated, flexible strut electrode 1332 and a plurality of flexible spot electrodes 1334. As shown, flexible spot electrodes 1334 are smaller than flexible strut electrode 1332. In this embodiment, tip electrode array 1302 also includes a split tip electrode 1340 having four selectively activatable (e.g., in unison or independently of one another) quadrant electrodes 1342. Alternatively, tip electrode array 1302 may include one or more electrodes in any suitable arrangement. For example, in some embodiments, tip electrode array 1302 includes a single electrode.

In regards to first spline 1320, for ablation applications, ring electrodes 1322 may be activated in unison. Further, for mapping applications, ring electrodes 1322 may be activated independent of one another. For second spline 1330, for ablation applications, flexible strut electrode 1332 may be activated without activating flexible spot electrodes 1334 (or, alternatively, flexible strut electrode 1332 and one or more flexible spot electrodes 1334 may be activated in unison). For mapping applications, flexible spot electrodes 1334 may be activated independent of one another (e.g., without activating flexible strut electrode 1332).

Those of skill in the art will appreciate that first spline 1320 and second spline 1330 are shown as included in the same embodiment as an example, and for ease of illustration. That is, in some embodiments, a basket catheter assembly may include one or more first splines 1320 and no second splines 1330. In other embodiments, a basket catheter assembly may include one or more second splines 1330 and no first splines 1320. Further, in yet other embodiments, a basket catheter assembly may include a combination of one or more first splines 1320 and one or more second splines 1330 (e.g., as in catheter assembly 1300).

In the embodiments described herein, applying energy between one or more pairs of individual electrodes on the tip of the device generally results in lesions with a more uniform area. This uniformity in area can also be accomplished by applying energy between i) some or all of the electrodes on the tip and ii) a body or intracardiac return electrode.

Those of skill in the art will appreciate that the various embodiments of catheter assemblies described herein may be implemented independently from one another or in any suitable combination.

The embodiments described herein provide a catheter assembly. The catheter assembly includes a tip electrode array including at least one tip electrode, the tip electrode array located at a distal end of the catheter assembly, and a mini electrode array including a plurality of mini electrodes, the mini electrode array positioned proximal of the tip electrode array, wherein each of the at least one tip electrode and each of the plurality of mini electrodes are configured to be activated independent of one another for mapping applications, and wherein at least some of the at least one tip electrode and the plurality of mini electrodes are configured to be activated in unison for ablation applications.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A catheter assembly comprising:
a tip electrode array comprising at least one tip electrode, the tip electrode array located at a distal end of the catheter assembly; and
a mini electrode array comprising a plurality of mini electrodes, the mini electrode array positioned proximal of the tip electrode array, wherein each of the at least one tip electrode and each of the plurality of mini electrodes are configured to be activated independent of one another for mapping applications, and wherein at least some of the at least one tip electrode and the plurality of mini electrodes are configured to be activated in unison for ablation applications.

2. The catheter assembly of claim 1, wherein the plurality of mini electrodes comprise a plurality of rectangular electrodes arranged in a brick-like pattern.

3. The catheter assembly of claim 1, wherein the plurality of mini electrodes comprise a plurality of circular electrodes.

4. The catheter assembly of claim 1, further comprising an annular electrode defining a plurality of apertures, wherein the plurality of mini electrodes are positioned in the plurality of apertures.

5. The catheter assembly of claim 1, wherein the at least one tip electrode comprises a plurality of tip electrodes, and wherein the tip electrode assembly further comprises a plurality of non-conductive segments that separate the plurality of tip electrodes.

6. The catheter assembly of claim 1, wherein the mini electrode array comprises four mini electrodes arranged in a square grid pattern.

7. The catheter assembly of claim 1, further comprising a ring electrode.

8. The catheter assembly of claim 7, wherein the plurality of mini electrodes are formed on the ring electrode.

9. The catheter assembly of claim 1, wherein the tip electrode array has one of a domed shape, a flat shape, a spherical shape, and a basket shape.

10. The catheter assembly of claim 1, wherein the catheter assembly is a basket catheter assembly comprising a plurality of splines, and wherein the mini electrode array comprises at least one of i) a plurality of ring electrodes on a single spline and ii) an elongated flexible strut electrode and a plurality of flexible spot electrodes on a single spline.

11. An electroporation system comprising:
a generator; and
a catheter coupled to the generator, the catheter comprising:
a handle;
a shaft extending distally from the handle; and
a catheter assembly coupled to a distal end of the shaft, the catheter assembly comprising:
a tip electrode array comprising at least one tip electrode, the tip electrode array located at a distal end of the catheter assembly; and
a mini electrode array comprising a plurality of mini electrodes, the mini electrode array positioned proximal of the tip electrode array, wherein each of the at least one tip electrode and each of the plurality of mini electrodes are configured to be activated independent of one another for mapping applications, and wherein at least some of the at least one tip electrode and the plurality of mini electrodes are configured to be activated in unison for ablation applications.

12. The system of claim 11, wherein the plurality of mini electrodes comprise a plurality of rectangular electrodes arranged in a brick-like pattern.

13. The system of claim 11, wherein the plurality of mini electrodes comprise a plurality of circular electrodes.

14. The system of claim 11, further comprising an annular electrode defining a plurality of apertures, wherein the plurality of mini electrodes are positioned in the plurality of apertures.

15. The system of claim 11, wherein the at least one tip electrode comprises a plurality of tip electrodes, and wherein the tip electrode assembly further comprises a plurality of non-conductive segments that separate the plurality of tip electrodes.

16. The system of claim 11, wherein the catheter is a linear catheter.

17. The system of claim 11, wherein the catheter is a loop catheter.

18. An ablation system comprising:
a console;
at least one catheter;
a cable system coupling the console to the at least one catheter;
a hub coupled between the console and the at least one catheter;
a first generator coupled to the hub, the first generator configured to deliver a first type of ablation energy to the at least one catheter via the hub; and
a second generator coupled to the hub, the second generator configured to deliver a second type of ablation energy to the at least one catheter via the hub, wherein the console is configured to selectively control the delivery of the first and second types of ablation energy to the at least one catheter via the hub.

19. The ablation system of claim 18, wherein the first generator is a radiofrequency generator, and wherein the second generator is a pulsed field ablation generator.

20. The ablation system of claim 18, further comprising a mapping system coupled to the at least one catheter through the cable system.
